(19) United States
(12) Patent Application Publication
(10) Pub. No.: US 2024/0047020 A1
(43) Pub. Date: Feb. 8, 2024

(54) EMPIRICAL OPTIMIZATION OF CONCRETE RECIPES

(71) Applicant: X Development LLC, Mountain View, CA (US)

(72) Inventors: Ray Jr. Anthony Nagatani, San Francisco, CA (US); Brian Howell, Berkeley, CA (US); Antonio Raymond Papania-Davis, Oakland, CA (US); Weishi Yan, Oakland, CA (US); Charles Stephen Spirakis, Mountain View, CA (US); Shijian Jin, Cambridge, MA (US)

(21) Appl. No.: 17/882,250

(22) Filed: Aug. 5, 2022

(51) Int. Cl. G16C 60/00; C04B 40/00

(52) U.S. Cl. CPC G16C 60/00; C04B 40/0032

(57) ABSTRACT

Methods, systems, and apparatus for developing recipes for concrete mixtures are disclosed. A method includes obtaining second input data including a second recipe for combining the plurality of ingredients; mixing the plurality of ingredients according to the second recipe to produce a second mixture; obtaining sensor data representing one or more qualities of the second mixture; evaluating the second mixture using the sensor data to obtain performance measures of the concrete mixture; processing the input data with the mixture prediction model to obtain a corresponding output of the mixture prediction model, the corresponding output including predicted performance measures for the second mixture; and adjusting parameters of the mixture prediction model based on comparing the output of the mixture prediction model to the performance measures of the second mixture.

### TECHNICAL FIELD

This specification relates generally to processes, methods, and systems for developing recipes for liquid suspensions.

### BACKGROUND

Many industrial processes rely upon suspensions of particles in a fluid state, ranging from ink resins for 3D printing, recipe formulation for food engineering and processing, suspensions for powder injection molding, drilling fluids carrying cuttings in oil and gas and mining exploration, and casting concrete for construction. In such applications, accurate prediction of the rheological behaviors of the suspension can be used to optimize processes to transport/deliver the suspension (e.g., reduce required pressure to pump) or modify the behavior of the fluid suspension to improve its performance in use (e.g., increase carrying capacity for drilling fluids, reduce/increase workability of concrete). However, as the complexity of suspensions increases (e.g., the number of unique types of particles within the suspensions increase), it becomes more challenging to accurately predict their rheological properties due to the increase in the number of different interactions which can occur. Multiscale material like concrete (e.g., coarse aggregate suspension in a mortar matrix, which is a suspension of sand particles in cement paste, which is a suspension of cement molecules in water) contains many different types of particles from micro- to macro-scale.

Many industrial processes involving suspensions of particles rely on either simple, well characterized baseline formulations that undergo extensive testing and evaluation, or real-time measurement of rheological properties to inform operational adjustments (e.g., adding admixtures and water to concrete systems after testing slump, adding rheology modifiers to drilling fluid). Computational methods such as high fidelity simulation may also be used to make predictions about fluid properties. When using operational modification, evaluation is low-throughput, situational, and reliant upon the expertise of the operator in the field.

### SUMMARY

In general, this disclosure relates to a process and system for optimizing recipes for concrete mixtures. The recipes can be adjusted based on characteristics of aggregate particles such as size, shape, and surface texture. The disclosed techniques can be used to predict performance of a mixture of particles using empirical models and machine learning techniques. Various concrete recipes can be mixed and evaluated using performance measures. A machine learning model can be trained to predict concrete mixtures based on the performance measures for the various concrete recipes. The disclosed techniques can be used in any context for predicting performance of liquid suspensions.

Concrete ingredients are characterized by a particle analyzer that employs various sensors to detect characteristics such as particle size, particle size distribution, particle shape, particle surface texture, porosity, chemical composition, and/or particle surface area. Concrete ingredients may be characterized by other analyzers using sensors that determine properties of the suspending fluid, such as pH, temperature, and/or viscosity. The concrete ingredients can be added incrementally to a mixture according to a recipe. The recipe can define amounts, e.g., absolute amounts, relative amounts, or both, of each ingredient of the mixture. Measurements of the mixture can be taken during the mixing process and after the mixing process. The measurements can be used to determine fluid behavior characteristics of the mixture and to evaluate performance measures of the mixture. The performance measures can include measures of slump, strength, water demand, rheology/viscosity, and flowability.

Different concrete mixtures can be produced using various types of particles, various recipes, and under various environmental conditions. Empirical data can be obtained indicating performance of the different mixtures. A mixture prediction model can be trained using the empirical data. An example mixture prediction model can be a deterministic machine learning model, such as a neural network, or a probabilistic machine learning model such as a Gaussian process. To train the mixture prediction model, the mixture prediction model can receive, as input, characterization data, environmental data, and recipe data for a particular mixture. The mixture prediction model can produce, as output, predicted performance of the particular mixture. Parameters of the mixture prediction model can be adjusted based on comparing the predicted performance to the performance measures determined for the particular mixture.

A trained mixture prediction model can be used to obtain a concrete recipe. For example, input data can be provided to the mixture prediction model. The input data can include characterizations of available ingredients, environmental data specifying environmental conditions in which the ingredients are combined, and recipe data defining a proposed recipe for a mixture. The mixture prediction model can process the input data to provide output data indicating predicted performance of the mixture. The recipe can then be iteratively adjusted based on the predicted performance. For each iteration, the mixture prediction model can determine an updated predicted performance. The iterations can continue until the predicted performance satisfies performance criteria. When the predicted performance satisfies performance criteria, the system can output a final recipe.

A concrete mixture can be mixed using the final recipe. Performance tests can then be performed on the concrete mixture. Results of the performance tests can be provided to the machine learning model. Parameters of the mixture prediction can be adjusted based on the performance tests. In this way, the mixture prediction model can be tuned for various aggregate particle characteristics, additives, environmental conditions, and recipes, improving accuracy of the mixture prediction model.

In general, innovative aspects of the subject matter described in this specification can be embodied in a method of training a mixture prediction model, including actions of: obtaining input data including: characterization data indicating characterizations of a plurality of ingredients, environmental data indicating environmental conditions in which the plurality of ingredients are combined, and recipe data defining a recipe for combining the plurality of ingredients; mixing the plurality of ingredients according to the recipe to produce a concrete mixture; obtaining sensor data representing one or more qualities of the concrete mixture; evaluating the concrete mixture using the sensor data to obtain performance measures of the concrete mixture; processing the input data with a mixture prediction model to obtain a corresponding output of the mixture prediction model, the corresponding output including predicted performance measures for the concrete mixture; and adjusting parameters of the mixture prediction model based on comparing the output of the mixture prediction model to the performance measures of the concrete mixture.

These and other embodiments may each optionally include one or more of the following features, alone or in combination. In some implementations, mixing the plurality of ingredients according to the recipe to produce the concrete mixture includes: determining, based on the recipe, a flow rate and a time for adding a first ingredient of the plurality of ingredients into a mixing vessel; and controlling a flow of the first ingredient into the mixing vessel based on the determined flow rate and time.

In some implementations, mixing the plurality of ingredients according to the recipe to produce the concrete mixture includes: determining, based on the recipe, a volume of a first ingredient to add to the concrete mixture; and controlling a flow of the first ingredient to add the determined volume of the first ingredient to a mixing vessel.

In some implementations, the method includes obtaining second input data including a second recipe for combining the plurality of ingredients; mixing the plurality of ingredients according to the second recipe to produce a second mixture; obtaining sensor data representing one or more qualities of the second mixture; evaluating the second mixture using the sensor data to obtain performance measures of the concrete mixture; processing the input data with the mixture prediction model to obtain a corresponding output of the mixture prediction model, the corresponding output including predicted performance measures for the second mixture; and adjusting parameters of the mixture prediction model based on comparing the output of the mixture prediction model to the performance measures of the second mixture.

In some implementations, obtaining sensor data representing one or more qualities of the concrete mixture includes obtaining sensor data at time intervals during mixing of the concrete mixture.

In some implementations, the environmental data indicates time-varying environmental conditions in which the plurality of ingredients are combined.

In some implementations, the recipe indicates an amount of each ingredient of the plurality of ingredients to be mixed.

In some implementations, the amount of each ingredient of the plurality of ingredients to be mixed includes a proportion, a weight, volume, or a flow rate.

In some implementations, the performance measures include at least one of slump or yield stress.

In some implementations, the ingredients include particles, the characterizations including at least one of: particle size, particle shape, particle surface texture, porosity, particle chemical composition, and particle surface area.

In some implementations, the plurality of ingredients include at least one of cement, water, fly ash, slag, plasticizer, fine aggregate, admixture, additives, and coarse aggregate.

In some implementations, the environmental conditions include at least one of temperature, humidity, and time.

In general, innovative aspects of the subject matter described in this specification can be embodied in A method of generating a recipe for a concrete mixture, including: obtaining input data including: characterization data indicating characterizations of a plurality of ingredients, environmental data indicating environmental conditions in which the plurality of ingredients are combined, and recipe data defining a recipe for combining the plurality of ingredients to produce a concrete mixture; predicting performance of the concrete mixture by processing the input data using a mixture prediction model configured to predict performance of the concrete mixture; iteratively adjusting the recipe and predicting performance of the concrete mixture until the predicted performance satisfies performance criteria to obtain a final recipe; and outputting the final recipe.

These and other embodiments may each optionally include one or more of the following features, alone or in combination. In some implementations, the method includes mixing a concrete mixture using the final recipe; and evaluating performance of the concrete mixture using one or more performance tests.

In some implementations, the ingredients include particles, the characterizations including at least one of: particle size, particle shape, particle surface texture, porosity, particle chemical composition, and particle surface area.

In some implementations, the recipe indicates an amount of each ingredient of the plurality of ingredients to be mixed.

In some implementations, the amount of each ingredient of the plurality of ingredients to be mixed includes a proportion, a weight, volume, or a flow rate.

In some implementations, predicting the performance of the concrete mixture includes predicting a slump or yield stress of the concrete mixture.

In some implementations, the plurality of ingredients include at least one of cement, water, fly ash, slag, plasticizer, fine aggregate, and coarse aggregate.

Other embodiments include corresponding computer systems, apparatus, computer program products, and computer programs recorded on one or more computer storage devices, each configured to perform the actions of the methods. A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

The details of one or more embodiments of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 depicts an exemplary concrete preparation system.

FIG. 2 depicts a block diagram of an exemplary control system for the concrete preparation system of FIG. 1.

FIG. 3 depicts an example process flow for developing recipes for concrete mixtures.

FIG. 4 depicts an example system for empirically mapping rheology characteristics to performance measures.

FIG. 5 depicts an example system for empirically determining rheology characteristics for aggregate mixtures.

FIG. 6 depicts an example system for training a mixture prediction model.

FIG. 7 depicts an example system for optimizing a concrete mixture recipe.

FIG. 8 is a flow diagram of an example process for training a mixture prediction model to predict performance of a mixture.

FIG. 9 is a flow diagram of an example process for developing a recipe for a mixture using a trained mixture prediction model.

FIG. 10 is a schematic diagram of a computer system that may be applied to any of the computer-implemented methods and other techniques described herein.

Like reference numbers and designations in the various drawings indicate like elements.

### DETAILED DESCRIPTION

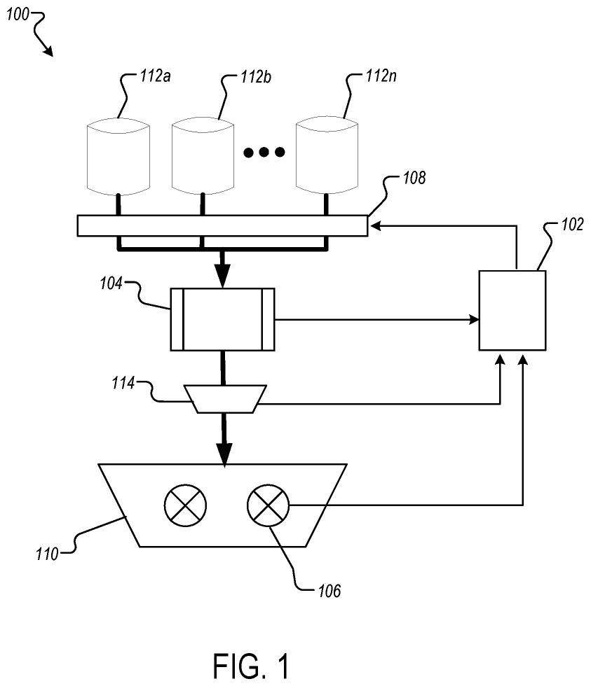

FIG. 1 depicts an exemplary concrete preparation system 100. In operation, concrete preparation system 100 measures characteristics of the raw ingredients of a concrete mixture. Concrete preparation system 100 can adaptively adjust the proportion of the raw ingredients added to the concrete mixture based on the measured characteristics to more accurately achieve desired structural properties in the final cured concrete. Using the disclosed techniques, rheological properties of concrete mixtures can be optimized by high throughput particle geometric characterization and machine learning accelerated fluid control.

Concrete preparation system 100 includes a control system 102. The control system 102 receives input from particle analyzing system 104 and concrete mix sensors 106. The control system 102 can control the operations of one or more ingredient metering systems 108 based on analyses of data obtained from one or both of the particle analyzing system 104 and concrete mix sensors 106, and based on modeling and simulation of aggregate mixes.

Concrete preparation system 100 includes raw ingredient storage bays or hoppers 112a-112n. The ingredient metering system 108 conveys the raw ingredients from the storage bays 112a-112n to a mixing vessel 110. For example, the ingredient metering system 108 can include a series of conveyors and augers to transfer raw ingredients from the storage bays 112a-112n into the mixing vessel 110. The concrete preparation system 100 differs from traditional concrete plants in that the raw ingredients are passed through the particle analyzing system 104 prior to delivery to the mixing vessel 110.

In some implementations, the ingredient metering system 108 may include a metering hopper 114 between the particle analyzing system 104 and the mixing vessel 110. The metering hopper 114 may be used to collect and measure (e.g., weigh) a raw ingredient as it passes through the particle analyzing system 104. For example, the weight of the ingredient measured by metering hopper 114 can be passed to the control system 102 permitting the control system to monitor the weight of the ingredient being measured in real-time. The control system 102 may then be able to make in-situ adjustments to how much of the ingredient to add to the concrete mixture based on real-time particle analysis of the ingredient from the particle analyzing system 104. In some implementations, concrete preparation system 100 can be retro-fit to a traditional ready-mix concrete plant. For example, adding the concrete preparation system 100 to a ready-mix plant may allow the ready-mix plant to more precisely tailor concrete mixes for specific applications and job sites.

The particle analyzing system 104 can include various different sensors configured to measure various characteristics of concrete ingredients. For example, the sensors used by the particle analyzing system 104 can include, but are not limited to, optical sensors (e.g., visible light cameras, infra-red cameras, dynamic optical microscopy sensors) and mechanical sensors (e.g., sieves, sedigraphs, impact hammer, electrodynamic vibrator). The measurement data is used by the control system 102 to determine characteristics of the ingredients of the concrete mixture. For example, ingredient characteristics can include, but are not limited to, particle sizes, particle shapes, surface areas, and particle sphericity.

The sensors of the particle analyzing system 104 are arranged to obtain measurement data of concrete ingredients as the ingredients are added to a concrete mixture. For example, in some implementations optical sensors can be arranged in an array along a conveyor or a chute used to convey the raw ingredients to the mixing vessel 110. The optical sensors can transmit images of the ingredients to the control system 102, which (as explained in more detail below) can use image processing algorithms to identify particle shapes and sizes.

Some implementations may include a series of sieves to separate particles of an ingredient by size. In such implementations, the optical sensors can be positioned proximate to each sieve to capture images of the particles passing through the sieve. The images can then be used, for example, to determine an approximate count of each size range of particles exiting each sieve. In such implementations, the separated particles may be recombined before being added to the mixing vessel 110.

The concrete mix sensors 106 provide rheometry measurements of the concrete mixture to the control system 102. For example, the concrete mix sensors 106 can measure various attributes of the concrete mixture that can be used to estimate or compute rheometric properties of the concrete mixture in real-time. The concrete mix sensors 106 can include, but are not limited to, viscosity sensors, rheometers, temperature sensors, moisture sensors, ultrasonic sensors (e.g., ultrasonic pulse velocity sensors), electrical property sensors (e.g., electrodes, electrical resistance probes), electromagnetic sensors (e.g., short-pulse radar), or other sensors (e.g., geophone, accelerometer). The concrete mix sensors 106 can include, but are not limited to, hydrophobicity, moisture content, XRD spectra, XRF spectra, static yield stress, acoustic impedance, p-wave speed, dynamic yield stress, static modulus of elasticity, Young's modulus, bulk modulus, shear modulus, dynamic modulus of elasticity (DME), Poisson's ratio, density, resonance frequency, nuclear magnetic resonance (NMR), dielectric constant, electric resistivity, polarization potential, and capacitance.

For example, viscosity, moisture, and temperature sensors can be installed in the mixing vessel 110. These sensors can be used to measure rheological properties of the concrete mixture such as changes in the viscosity of the mixture over time and at different moisture content levels and temperatures. As described in more detail below, the control system 102 can use the rheometry measurements to determine whether and how much additional ingredients should be added to the concrete mixture to obtain desired concrete properties.

Post-curing characteristics can be determined from known relationships, e.g., as indicated by multi-dimensional lookup tables relating experimentally obtained post-curing characteristics to mixtures with known properties, by applying theoretical and analytical particle packing model-based Bayesian optimization algorithms to the rheometry measurements, or a combination thereof.

In some examples, rheometry measurements can be performed on the initial concrete mixture. Rheometry measurements of the concrete mixture with the ingredients added can be estimated based on the measured characteristics of the ingredients. The rheometry measurements are used to predict workability of a mixture.

The actual rheometry measurements of the concrete mixture can be obtained and compared with the estimated rheometry to determine whether to add additional ingredients. The system can determine, based on the rheometry measurements, whether the concrete mixture is likely to satisfy workability constraints. The initial mixture can be adjusted through an iterative process until the rheometry measurements indicate that the concrete mixture is likely to achieve the desired workability.

During the iterative adjustment process, portions of concrete ingredients are incrementally added to the initial concrete mixture while changes in the rheometry measurements are monitored. Additional portions of ingredients are added until the rheometry measurements and the current recipe indicate that the concrete mixture is likely to achieve the desired workability and post-cure characteristics. Such post-curing characteristics can include, but are not limited to, compressive strength, tensile/flexural strength, flowability, toughness, cure time, cure profile, finish, density (wet & dry), thermal insulation, shrinkage, and slump.

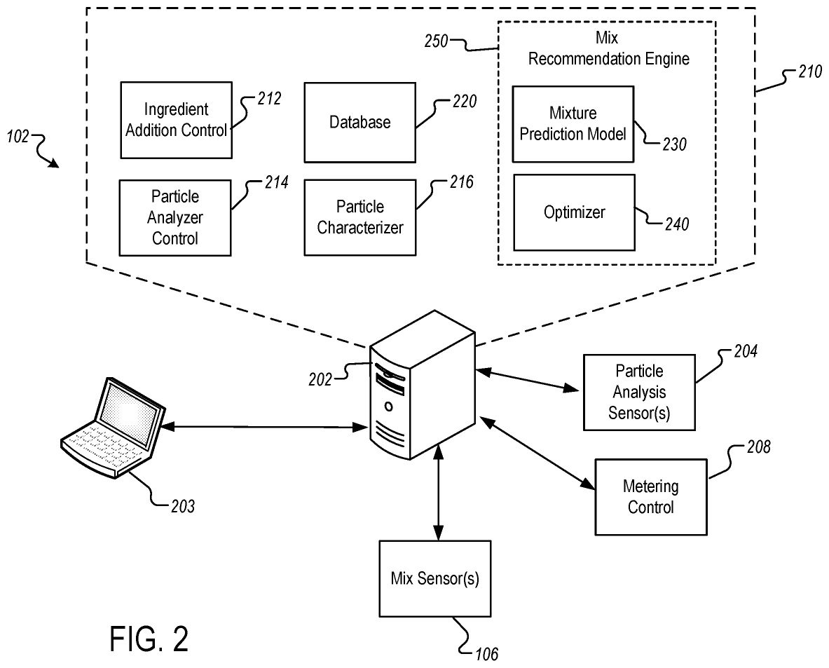

FIG. 2 is a block diagram of an exemplary control system 102 for the concrete preparation system 100. The control system 102 includes a computing system 202 in communication with the concrete mix sensors 106, particle analysis sensors 204 of the particle analyzing system 104, a metering control system 208 which can control operations of the ingredient metering system 108. Computing system 202 is configured to control various aspects of the concrete preparation process. For example, computing system 202 can store and execute one or more computer instruction sets to control the execution of aspects of the concrete preparation processes described herein. Computing system 202 can include a system of one or more computing devices. The computing devices can be, e.g., a system of one or more servers. For example, a first server can be configured to receive and process data from the concrete mix sensors 106 and the particle analysis sensors 204. Another server can be configured to interface with the metering control system 208 and issue control commands based on analysis results from the first server.

In some implementations, the computing system 202 can be operated or controlled from a user computing device 203. User computing device 203 can be a computing device, e.g., desktop computer, laptop computer, tablet computer, or other portable or stationary computing device.

Briefly, computing system 202 can control the overall concrete preparation system 100 to prepare concrete mixtures. The computing system 202 can use the particle analysis sensors 204 to characterize concrete ingredients as they are added to a concrete mixture.

The computing system 202 obtains rheometry measurements from the mix sensors 106 as the concrete mixture is mixed in the mixing vessel 110. The system compares the rheometry measurements with estimated rheometry measurements to determine, e.g., whether the concrete mixture will meet desired post-curing mechanical properties or whether additional ingredients should be added.

In some implementations, computing system 202 can include a set of operations modules 210 for controlling different aspects of a concrete additive manufacturing process. The operation modules 210 can be provided as one or more computer executable software modules, hardware modules, or a combination thereof. For example, one or more of the operation modules 210 can be implemented as blocks of software code with instructions that cause one or more processors of the computing system 202 to execute operations described herein. In addition or alternatively, one or more of the operations modules can be implemented in electronic circuitry such as, e.g., programmable logic circuits, field programmable logic arrays (FPGA), or application specific integrated circuits (ASIC). The operation modules 210 can include an ingredient addition controller 212, a particle analyzer controller 214, one or more databases 220, a particle characterizer 216, a mixture prediction model 230, an optimizer 240,

Ingredient addition controller 212 interfaces with the metering control system 208 to control the addition of ingredients to the concrete mixing vessel 110. For example, the ingredient addition controller 212 can issue commands from the computing system 202 to the metering control system 208 to control the addition of ingredients to the concrete mixture in the mixing vessel 110.

Particle analyzer control 214 interfaces with the particle analysis sensors 204 of the particle analyzing system 104. Particle analyzer controller 214 receives and buffers data from the particle analysis sensors 204. The particle analyzer controller 214 can process the sensor data to determine particle characteristics of each analyzed ingredient. For example, the particle analyzer controller 214 can execute data analysis algorithms to interpret the sensor data and determine particle characteristics including, but not limited to, particle sizes, particle shapes, and particle surface areas.

The computing system 202 includes a mix recommendation engine 250. The mix recommendation engine 250 includes a mixture prediction model 230 and an optimizer 240. The control system can employ mixture prediction model 230 to estimate the rheometry parameters of a given concrete mixture based on the particle characteristics of the ingredients.

In some implementations, the mixture prediction model 230 can include a machine learning model to estimate rheometry parameters for a concrete mixture from measured particle characteristics. For example, the machine learning model can include a model that has been trained on experimental data to receive particle characteristics of concrete ingredients as input, and to generate a predicted output. In some implementations, the machine learning model is a deep learning model that employs multiple layers of models to generate an output for a received input. A deep neural network is a deep machine learning model that includes an output layer and one or more hidden layers that each applies a non-linear transformation to a received input to generate an output. In some cases, the neural network may be a recurrent neural network. A recurrent neural network is a neural network that receives an input sequence and generates an output sequence from the input sequence. In particular, a recurrent neural network uses some or all of the internal state of the network after processing a previous input in the input sequence to generate an output from the current input in the input sequence. In some other implementations, the machine learning model is a convolutional neural network. In some implementations, the machine learning model is an ensemble of models that may include all or a subset of the architectures described above.

In some implementations, the machine learning model can be a feedforward autoencoder neural network. For example, the machine learning model can be a three-layer autoencoder neural network. The machine learning model may include an input layer, a hidden layer, and an output layer. In some implementations, the neural network has no recurrent connections between layers. Each layer of the neural network may be fully connected to the next, there may be no pruning between the layers. The neural network may include an ADAM optimizer, or any other multi-dimensional optimizer, for training the network and computing updated layer weights. In some implementations, the neural network may apply a mathematical transformation, such as a convolutional transformation, to input data prior to feeding the input data to the network.

In some implementations, the machine learning model can be a supervised model. For example, for each input provided to the model during training, the machine learning model can be instructed as to what the correct output should be. The machine learning model can use batch training, training on a subset of examples before each adjustment, instead of the entire available set of examples. This may improve the efficiency of training the model and may improve the generalizability of the model. The machine learning model may use folded cross-validation. For example, some fraction (the “fold”) of the data available for training can be left out of training and used in a later testing phase to confirm how well the model generalizes. In some implementations, the machine learning model may be an unsupervised model. For example, the model may adjust itself based on mathematical distances between examples rather than based on feedback on its performance.

A machine learning model can be trained to estimate rheometry parameters for concrete mixtures based on measured characteristics of the ingredients to the mixture. In some examples, the machine learning model can be trained on experimentally determined data relating known characteristics of concrete ingredients to experimentally determined rheometry parameters.

The computing system 202 can store one or more databases 220 that correlate different measured parameters to experimentally determined characteristics of a concrete mixture or post-curing concrete. In some examples, the database 220 correlates concrete particle characteristics to experimentally determined rheometry parameters. For example, the database 220 can include a lookup table correlating desired post-curing concrete characteristics to concrete mixture rheometry parameters, a lookup table correlating ingredient characteristics to particle packing efficiencies, and a lookup table correlating ingredient characteristics to mixture remoter parameters. Each lookup table can be a multi-dimensional data structure containing measurable concrete parameters, concrete mixture parameters, or ingredient characteristics to experimentally determined parameters.

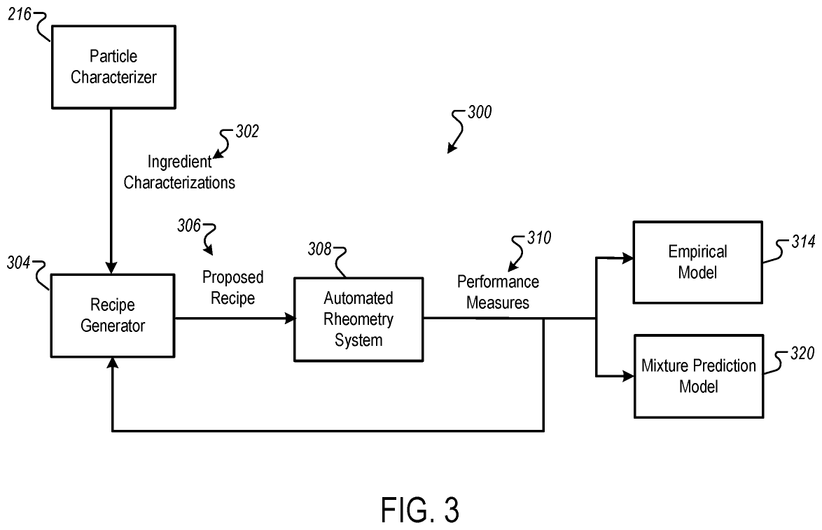

FIG. 3 depicts an example system 300 for generating models for developing recipes for concrete mixtures. The system 300 can be implemented to develop a database of mixture models using low cost, high throughput experimental techniques. The system 300 includes a recipe generator 304 and an automated rheometry system 308. The system produces an empirical model 314 and a mixture prediction model 320.

Concrete ingredients are characterized by a particle characterizer 216 that employs particle analysis sensors 204 to generate characterizations 302 of ingredients available for a concrete mixture. The ingredients can include, for example, fine aggregate particles and coarse aggregate particles. The characterizations 302 can include any combination of the following ingredient properties. The characterizations 302 can include particle size, particle size distribution, particle shape, particle surface texture, particle gradations, porosity, chemical composition, and/or particle surface area. The characterizations 302 can include dimensions, elongation, flatness, lengths of three axes, volume, angularity, sphericity, and aspect ratio. The characterizations 302 can include pH, temperature, and viscosity. The characterizations 302 can include spatial characterizations obtained using a laser scanner or camera. The characterizations 302 can include porosity or texture obtained using spectroscopy. The characterizations 302 can include chemical composition obtained using Fourier transform infrared spectroscopy (FTIR), X-ray powder diffraction (XRD), or X-ray fluorescence (XRF). The characterizations 302 can include results of differential thermal analysis.

The recipe generator 304 receives, as input, the characterizations 302 of the ingredients. The recipe generator 304 generates a proposed recipe 306 for a mixture of ingredients using the characterizations 302. The proposed recipe 306 can define amounts of each ingredient of the mixture. The amounts of ingredients can be, for example, absolute amounts, relative amounts, or both, of each ingredient of the mixture.

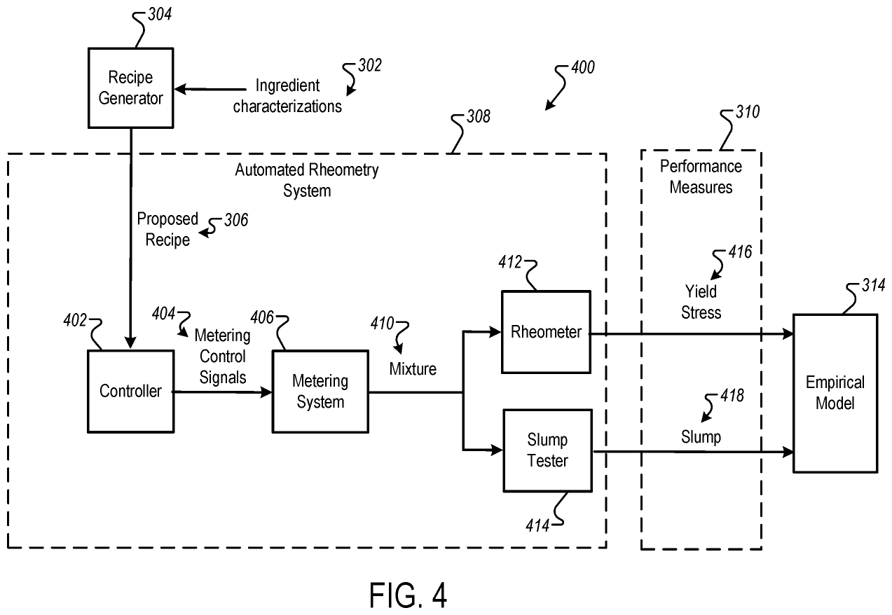

The proposed recipe generator 304 outputs the proposed recipe 306 to an automated rheometry system 308. The automated rheometry system 308 includes equipment for creating a mixture and for taking measurements of the mixture. Operations of the automated rheometry system are described in greater detail with reference to FIGS. 4, 5, and 6.

The automated rheometry system 308 outputs performance measures 310 of the mixture. Performance measures 310 can include, for example, yield stress and slump of the mixture. The performance measures 310 are used as input to an empirical model 314 and a mixture prediction model 320.

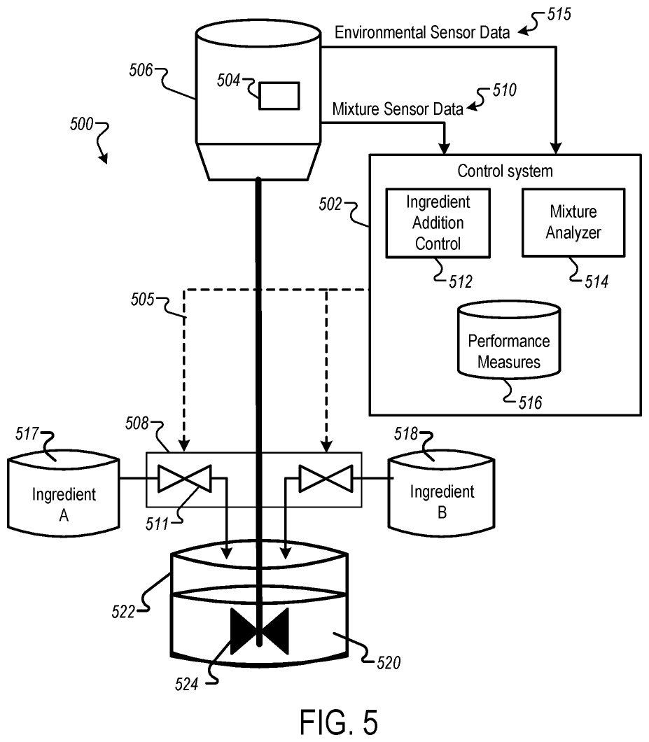

The empirical model 314 is a low data driven model that is used to correlate yield stress to slump. Operations of the empirical model 314 are described in greater detail with reference to FIG. 4. The mixture prediction model 320 is a high data driven model that is used to predict rheological characteristics of mixtures based on characterizations of ingredients. Operations of the mixture prediction model 320 are described in greater detail with reference to FIGS. 5, 6, and 7.

FIG. 4 depicts an example system 400 for empirically mapping rheology characteristics to performance measures. The system 400 uses feedback control mechanisms and known relationships between rheological properties (e.g., relationships between yield stress and slump) to enable rheological characterization of suspensions. For example, yield stress of a mixture can be measured by a rheometer. Interpolation can be used to predict rheological properties such as slump of a mixture. The system 400 can be implemented to improve efficiency of determining rheological measurements of suspensions.

Referring to FIG. 4, the automated rheometry system 308 includes a controller 402, a metering system 406, a rheometer 412, and a slump tester 414. The recipe generator 304 outputs a proposed recipe 306 to the automated rheometry system 308.

The controller 402 receives the proposed recipe 306 as input. The controller 402 outputs metering control signals 404 to control operations of a metering system 406 according to the proposed recipe 306. For example, the metering control signals 404 can control settings of flow control valves in order to control a flow rate of ingredients into a mixing vessel. The metering system 406 conveys ingredients from storage bays to a mixing vessel. The metering system 406 thus produces a mixture 410 based on the proposed recipe 306.

The mixture 410 is tested using the rheometer 412 and the slump tester 414. The rheometer 412 can be, for example, an ICAR rheometer. A rheometer is a device used to measure the way in which a dense fluid (e.g., a liquid, a suspension, a slurry) flows in response to applied forces. Rheometers are used for fluids which cannot be defined by a single value of viscosity and therefore require more parameters to be set and measured than is the case for a viscometer. Rheometers measure the rheology of fluids. The rheometer 412 is configured to characterize static yield stress, dynamic yield stress, and plastic viscosity of mixtures. The rheometer 412 outputs the measured yield stress 416 of the mixture 410.

The slump tester 414 determines a slump of the mixture using a slump test. The slump test is a test of wet flowability, and can be used as an indicator of industrial usefulness of the mixture 410. The result of the slump test is a measure in the distance of slump from an original cone height to a released slumped state. Different applications require different degrees of slump from very stiff (low slump) to more liquid (higher slump). In general, the wet flowability rating is a range from zero to twelve inches. The slump tester 414 outputs the measured slump 418 of the mixture 410.

The performance measures 310 are used to develop an empirical model 314. To generate the empirical model 314, regression can be used to find proportional relationships between yield stress 416 and slump. In some examples, the empirical model 314 includes a mapping of yield stress 416 to slump.

The empirical model 314 can be developed using performance measures 310 from multiple different mixtures. In an example, the rheometer 412 and the slump tester 414 measure six different mixtures in order to develop a baseline empirical model. To develop the six different mixtures, the proposed recipe 316 can be modified with five different water to binder (WB) ratios. For each mixture, the yield stress 416 and the slump 418 can be measured.

The resulting empirical model 314, including the mapped correlation between yield stress to slump, can be used as a translator between measured characteristics and expected performance. Given a mixture with a particular measured yield stress, the empirical model 314 can be used to predict the slump of the mixture. For example, a rheometer can be integrated with a cement mixing truck. The rheometer can measure the yield stress of a mixture in the cement mixing truck. The empirical model 314 can then be used to monitor the predicted slump of the mixture as the mixture is being transported in the cement mixing truck.

FIG. 5 depicts an example automated rheometry system 500 for empirically determining rheology characteristics for aggregate mixtures. Using the system 500, different concrete mixtures are produced using various types of particles, various recipes, and under various environmental conditions. Empirical data is obtained indicating performance of the different mixtures.

The system 500 can be used to determine correlations between properties of ingredients and rheological properties of suspensions composed of the ingredients. Example properties of ingredients can include spatial properties of particles such as surface area per unit weight. Rheological properties of suspensions can include a yield stress of a mixture composed of cement paste and varying concentrations of aggregates.

The system 500 includes a rheometer 506, a mixing vessel 522, a metering system 508, and storage hoppers 517, 518. In general, using the system 500, ingredients are mixed according to a recipe to produce a mixture 520, while measurements of the mixture are obtained using a rheometer 506. The rheometer 506 obtains real time measurements of the mixture 520 as the mixture 520 is mixed by mixing blades 524 in the mixing vessel 522.

In the example of FIG. 5, storage hopper 517 contains Ingredient A. Storage hopper 518 contains Ingredient B. In some examples, Ingredients A and B can each be a type of aggregate. For example, Ingredient A may be fine aggregate, and Ingredient B may be coarse aggregate. Although shown in FIG. 5 as having only two ingredients, mixtures can include many different ingredients. Ingredients can include, for example, aggregate particles, cement, water, fly ash, and slag. Ingredients can include admixture and additives, such as boric acid, H3PO4, amine, or any combination of these. Ingredients are mixed by controlling a flow of each ingredient into a mixing vessel 522.

The control system 502 includes an ingredient addition control system 512, a mixture analyzer 514, and a database of performance measures 516. The ingredient addition control system 512 generates control signals 505 for controlling the metering system 508. The control signals 505 control the amount of each ingredient added to the mixing vessel 522, e.g., by adjusting positions of one or more valves 511 of the metering system 508. The positions of the valves 511 can be, for example, open, shut, or partially open. The control signals 505 can control a degree of opening of the valves 511 in order to adjust the flow rate of ingredients to the mixing vessel 522.

During operation of the system 500, the ingredient addition control system 512 generates control signals 505 for controlling the metering system 508 to add appropriate volumes of ingredients to the mixing vessel 522 according to an initial recipe. For example, the control signals 505 may direct the metering system 508 to add Ingredient A to the mixing vessel 522 at a first flow rate for a first duration of time. The control signals 505 may direct the metering system 508 to add Ingredient B to the mixing vessel 522 at a second flow rate for a second duration of time. In some implementations, the ingredient addition control system 512 can control an order in which the ingredients are added to the mixing vessel 522.

The rheometer 506 takes measurements of mixtures 520 in the mixing vessel 522, and generates sensor data using one or more sensors 504. The sensor data can be obtained during the mixing process, after the mixing process, or both. Sensors 504 can include any of the concrete mix sensors 106 described with reference to FIG. 1. Sensors 504 can include for example, thermocouples for measuring temperature, moisture sensors for measuring humidity, infrared carbon dioxide sensors for measuring carbon dioxide level, acoustic sensors for measuring air entrainment, FTIR/XRF sensors for measuring chemical composition, mixture water content sensors, or any combination of these.

The sensor data can include mixture sensor data 510 and environmental sensor data 515. The mixture sensor data 510 indicates qualities of the mixture 520 such as water content, yield stress, shear rate, torque, and shear stress. The environmental sensor data 515 indicates environmental conditions of the mixing vessel 522 such as temperature, pressure, and humidity. The rheometer 506 outputs the sensor data generated by the sensors 504 to a control system 502.

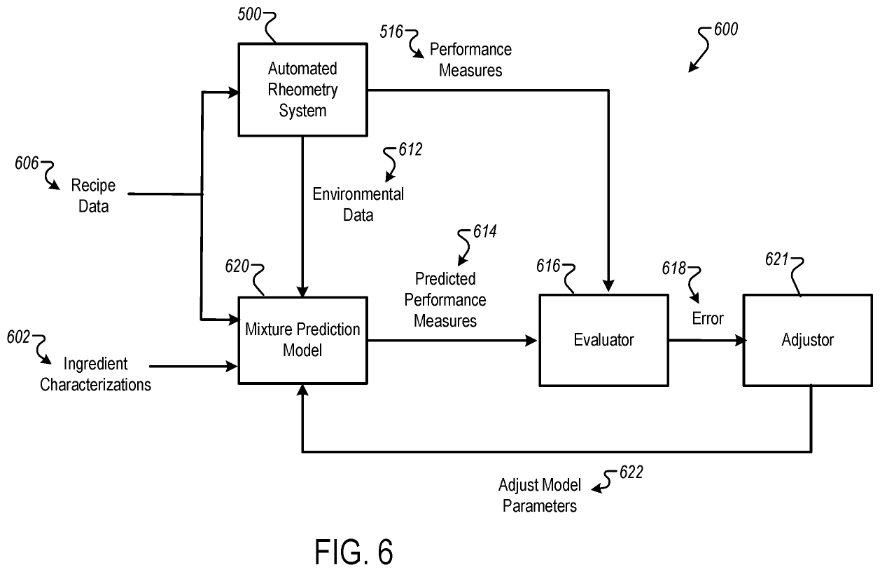

The mixture analyzer 514 uses the sensor data to determine rheological properties and fluid behavior characteristics of the mixture 520. The mixture analyzer 514 can analyze the mixture sensor data 510, the environmental sensor data 515, or both, to determine performance measures 516 representing the rheological properties of the mixture 520. The performance measures 516 can include measures of slump, strength, water demand, rheology/viscosity, and flowability. In some examples, the performance measures 516 can be stored in a database of performance measures 516. In some examples, the performance measures 516 can be used to train a mixture prediction model to predict performance of mixtures. A process of using performance measures 516 to train a mixture prediction model is described with reference to FIG. 6.

In some examples, sensor data is captured at sampling times. In some examples, the sampling times are at designated time intervals. In some examples, the sampling times are defined by a sampling rate. At each sampling time, mixture sensor data 510, environmental sensor data 515, or both, can be collected. The mixture analyzer 514 can analyze the mixture sensor data 510 to determine performance measures 516 at each sampling time. For each sampling time, performance measures indicating performance of the mixture at the sampling time, and environmental conditions of the mixing vessel 522 at the sampling time, can be stored in the database of performance measures 516.

During the mixing process, the ingredient addition control system 512 controls the rate of adding ingredients to the mixing vessel 522. In some examples, the amount of one or more ingredients in the mixture 520 can be incrementally increased over time while mixing is occurring and measurements are collected. For example, the ingredient addition control system 512 may control the metering system 508 to provide a particular volume of Ingredient A to the mixing vessel 522. The ingredient addition control system 512 may then control the metering system 508 to add Ingredient B to the mixing vessel 522 at a particular flow rate. The metering system 508 thus adds Ingredient B incrementally to the mixing vessel 522, as the mixture 520 is mixed by the mixing blades 524 and the sensors 504 of the rheometer 506 generate sensor data 510. The process may continue until the mixing blades 524 are no longer able to turn in the mixture 520.

After creating the mixture 520, the mixing vessel 522 can be emptied and a new mixture can be produced according to a new recipe. The new recipe may specify different ingredients than the initial recipe, different amounts of ingredients than the initial recipe, or both. The process can be repeated in order to obtain sensor data from mixtures including various combinations of ingredients. Using this process, the rheometer can be used to experimentally measure properties of mixtures of varied composition.

In some implementations, the control system 502 includes a proportional integral derivative (PID) controller. The PID controller uses a control loop feedback mechanism to control process variables. The PID controller can be implemented to adjust the control signals 505 to control an amount of each ingredient added to the mixing vessel while maintaining one or more parameters within specified bounds. For example, environmental sensor data 515 may be obtained indicating a temperature of the mixing vessel 522. The PID controller can adjust the control signals 505 to control ingredient addition to the mixing vessel 522 in order to maintain the temperature within specified limits.

The mixture analyzer 514 determines performance measures for various mixtures using the sensor data 510. The performance measures are stored in the database of performance measures 516. The performance measures 516 can be, for example, empirical rules establishing limits for flowability of various mixing proportions. In some examples, the performance measures 516 can include tarantula curves and/or coarseness factor charts. Since wet flowability ratings are not monotonic, the ratings can be plotted as isolines (e.g., contour lines) in a graph of different size aggregate proportions, called a tarantula curve. A tarantula curve can include a graph of percent retained aggregate vs. sieve size. A coarseness factor chart can be a graph of coarseness factor vs. workability. The performance measures 516 can establish static limits for upper and lower bounds for gradations.

FIG. 6 depicts an example system 600 for training a mixture prediction model 620. The mixture prediction model 620 can be trained to computationally predict rheological behavior of mixtures, e.g., using machine learning methods. Predicting mixture performance using the mixture prediction model is more computationally efficient than simulating multiscale suspensions.

The mixture prediction model 620 can be trained using empirical data, e.g., empirical data obtained using the automated rheometry system 500. An example mixture prediction model is a deterministic machine learning model, such as a neural network, or a probabilistic machine learning model such as a Gaussian process.

In general, the mixture prediction model 620 receives input data. The input data includes ingredient characterizations 602, environmental data 612, and recipe data 606 for a particular mixture. The mixture prediction model 620 produces, as output, predicted performance measures 614 of the particular mixture. Parameters of the mixture prediction model 620 can be adjusted based on comparing the predicted performance measures 614 to performance measures 516 determined by the automated rheometry system 500 for the particular mixture.

The ingredient characterizations 602 can include characterizations of multiple ingredients. Ingredients can include, for example, cement, water, fly ash, slag, admixture, additives, or any combination of these. The characterizations 602 can include any combination of the characterizations 302 described with respect to FIG. 3.

The recipe data 606 defines a recipe for combining the ingredients into a mixture, and indicates amounts of the ingredients in the mixture. The recipe data 606 can specify an amount of each ingredient of the mixture, e.g., example, an absolute amount, relative amounts, or both. In some examples, the recipe data 606 specifies weights of each ingredient to be added to the mixture.

The environmental data 612 indicates environmental conditions in which the ingredients are combined. In some examples, the environmental data 612 is collected during the mixing process and indicates time-varying conditions in a mixing vessel during the mixing process. For example, the environmental data 612 can include the environmental sensor data 515 representing real-time conditions in the mixing vessel 522. The environmental data 612 can indicate, for example, a time to steady state conditions, temperature, change in temperature, humidity, change in humidity, CO2 concentration, change in CO2 concentration, or any combination of these.

In some examples, the environmental data 612 indicates planned environmental conditions in which the ingredients are to be combined. For example, the environmental data 612 can include mixing vessel settings indicating upper and lower bounds for environmental parameters, e.g., temperature, pressure, and humidity.

The mixture prediction model 620 processes processing the input data and produces a corresponding output. The output includes predicted performance measures 614 for the mixture. An evaluator 616 compares the output predicted performance measures 614 to the performance measures 516 determined by the automated rheometry system 500 for the same mixture. The evaluator 616 determines an error 618 between the predicted performance measures 614 and the measured performance measures 516. The error 618 is output to an adjustor 621. The adjustor adjusts model parameters 622 of the mixture prediction model 620 based on the error 618.

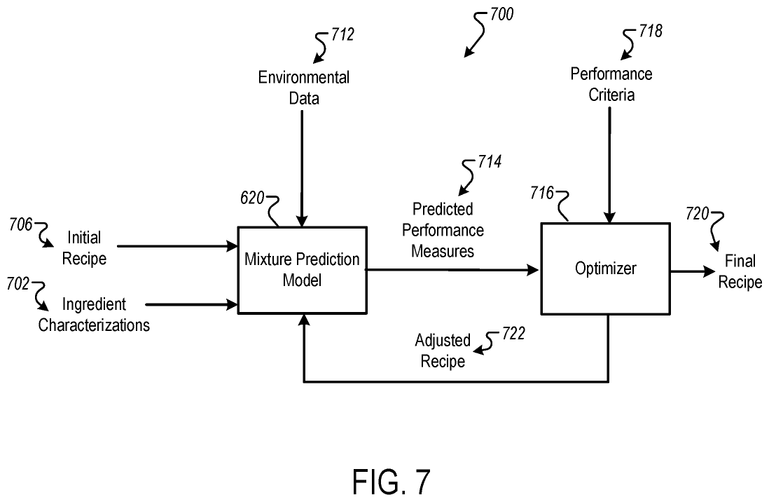

FIG. 7 depicts an example system 700 for optimizing a concrete mixture recipe. The system 700 can be used to create recipes that meet strength, workability, and durability requirements. In general, the mixture prediction model 620 is implemented to predict rheological properties from ingredient characterizations and mixture recipes. The predicted rheological properties are provided as an input to an optimization model that suggests ingredients that can alter the rheological properties of the suspension to meet desired features. For example, the optimization model, e.g., optimizer 716, may suggest addition of a particular admixture or rheology modifier based on the predicted performance measures and desired performance of the mixture. The optimizer 716 aims to optimize the post-cure performance by adding components that the model predicts will improve performance, subject to satisfying workability requirements.

The mixture prediction model 620 is trained as described with reference to FIG. 6. The trained mixture prediction model 620 receives input data. The input data includes ingredient characterizations 702, environmental data 712, and an initial recipe 706. The environmental data 712 specifies environmental conditions in which the ingredients are combined, or expected environmental conditions in which the ingredients are to be combined. The initial recipe 706 defines a proposed recipe for a mixture.

The mixture prediction model 620 processes the input data to provide output data indicating predicted performance measures 714 of the mixture. The optimizer 716 receives the predicted performance measures 714 as input. The optimizer 716 can employ one or more optimization algorithms. The optimizer 716 can use, for example, Bayesian optimization, Genetic algorithms, Covariance matrix adaptation-evolutionary strategies (CMAES), Particle swarm optimization (PSO), random search, or any combination of these.

In some examples, the optimizer can receive and/or store performance criteria 718. In some examples, the performance criteria 718 are received as user input. In some examples, the performance criteria 718 includes upper and lower limits of one or more performance measures. In some examples, the performance criteria include target values and tolerances for one or more performance measures.

The optimizer 716 compares the predicted performance measures 714 to the performance criteria 718. If the predicted performance measures 714 satisfy requirements of the performance criteria 718, the optimizer 716 outputs the initial recipe 706 as a final recipe 720. If the predicted performance measures 714 do not satisfy requirements of the performance criteria 718, the optimizer 716 can revise the recipe and provide an adjusted recipe 722 to the mixture prediction model 620.

The optimizer 716 proposes the adjusted recipe 722 based on the predicted performance measures 714. The mixture prediction model 620 then determines a predicted performance of the adjusted recipe 722. The process can repeat such that the recipe is iteratively adjusted based on the predicted performance. The iterations can continue until the optimizer 716 determines that the predicted performance satisfies performance criteria. When the predicted performance satisfies performance criteria, the system can output a final recipe 720. In this way, the optimizer 716 continues to generate adjusted recipes 722 until the predicted performance measures 714 satisfy the performance criteria 718

The optimizer 716 outputs the final recipe 720. The computing system 202 can use the final recipe 720 to adjust the ingredient addition controller 212. The ingredient addition controller 212 can then control addition of ingredients to the concrete mixing vessel 110 based on the final recipe 720. In this way, the final recipe 720 can be used to produce a concrete mixture that is expected to satisfy the performance criteria 718.

Performance tests can be performed on the concrete mixture produced using the final recipe 720. Results of the performance tests can be provided to the mixture prediction model 620. Parameters of the mixture prediction model 620 can be adjusted based on the performance tests. In this way, the mixture prediction model 620 can be tuned for various aggregate particle characteristics, additives, environmental conditions, and recipes, improving accuracy of the mixture prediction model 620 over time.

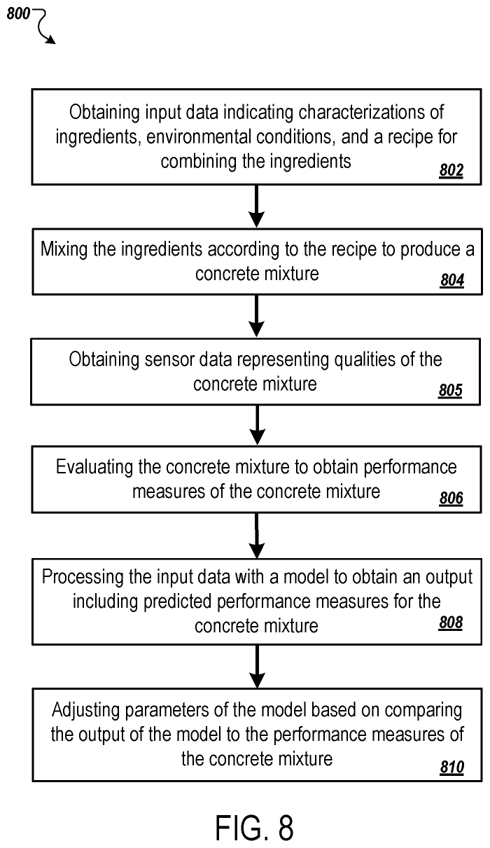

FIG. 8 is a flow diagram of an example process for training a mixture prediction model to predict performance of a mixture. The process 800 can be performed by one or more computing devices. For example, the process 800 may be performed by computing system 202 of FIG. 2.

The process 800 includes obtaining input data indicating characterizations of ingredients, environmental conditions, and a recipe for combining the ingredients (802). For example, referring to FIG. 6, the mixture prediction model 620 receives input data including ingredient characterizations 602, environmental data 612, and recipe data 606.

The process 800 includes mixing the ingredients according to the recipe (804). For example, referring to FIGS. 5 and 6, the automated rheometry system 500 mixes ingredients to form the mixture 520 in the mixing vessel 522 according to a recipe specified by the recipe data 606.

The process 800 includes obtaining sensor data representing qualities of the mixture (805). For example, referring to FIG. 5, the control system 502 obtains mixture sensor data 510 representing qualities of the mixture 520.

The process 800 includes evaluating the mixture to obtain performance measures of the mixture (806). For example, referring to FIG. 5, the mixture analyzer 514 evaluates the mixture 520 using the mixture sensor data 510 to obtain performance measures 516 of the mixture 520.

The process 800 includes processing the input data with a model to obtain an output including predicted performance measures (808). For example, referring to FIG. 6, the mixture prediction model 620 processes the ingredient characterizations 602, the environmental data 612, and the recipe data 606 to obtain output including the predicted performance measures 614.

The process 800 includes adjusting parameters of the model based on comparing the output of the model to the performance measures (810). For example, referring to FIG. 6, the adjustor 621 adjusts parameters 622 of the mixture prediction model 620 based on an error 618 between the predicted performance measures 614 and the performance measures 516,

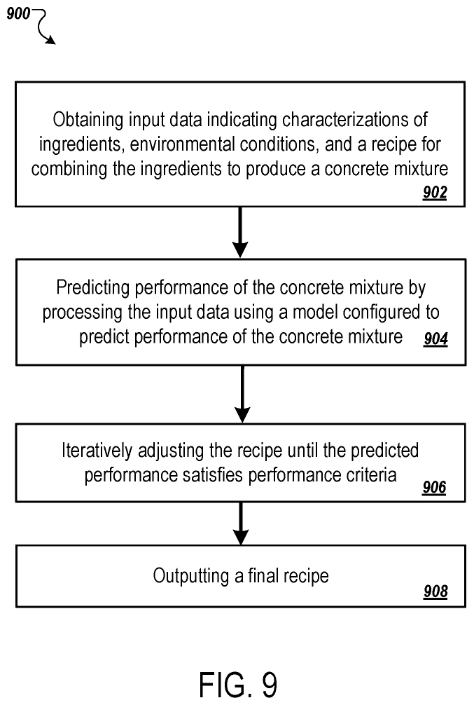

FIG. 9 is a flow diagram of an example process for developing a recipe for a mixture using a trained mixture prediction model. The process 900 can be performed by one or more computing devices. For example, the process 900 may be performed by computing system 202 of FIG. 2.

The process 900 includes obtaining input data indicating characterizations of ingredients, environmental conditions, and a recipe for combining the ingredients (902). For example, referring to FIG. 7, the mixture prediction model 620 receives input data including ingredient characterizations 702, environmental data 712, and an initial recipe 706.

The process 900 includes predicting performance of the mixture by processing the input data using a model configured to predict performance of the mixture (904). For example, referring to FIG. 7, the mixture prediction model 620 processes the ingredient characterizations 702, the environmental data 712, and the initial recipe 706 to obtain output including the predicted performance measures 714.

The process 900 includes iteratively adjusting the recipe until the predicted performance satisfies performance criteria (906). For example, referring to FIG. 7, the optimizer 716 iteratively generates adjusted recipes 722 until the predicted performance measures 714 satisfy the performance criteria 718.

The process 900 includes outputting a final recipe (908). For example, referring to FIG. 7, the optimizer 716 outputs the final recipe 720. The final recipe 720 can be used to produce a concrete mixture, e.g., using the concrete preparation system 100.

The processes 800, 900 can be implemented to predict the rheological properties of solids in a suspension or mixture based on physical properties of the solids. By establishing the connection between the physical characterization of a solid and its behavior as part of a population of solids in a mixture, end-state rheological properties of the mixture can be predicted.

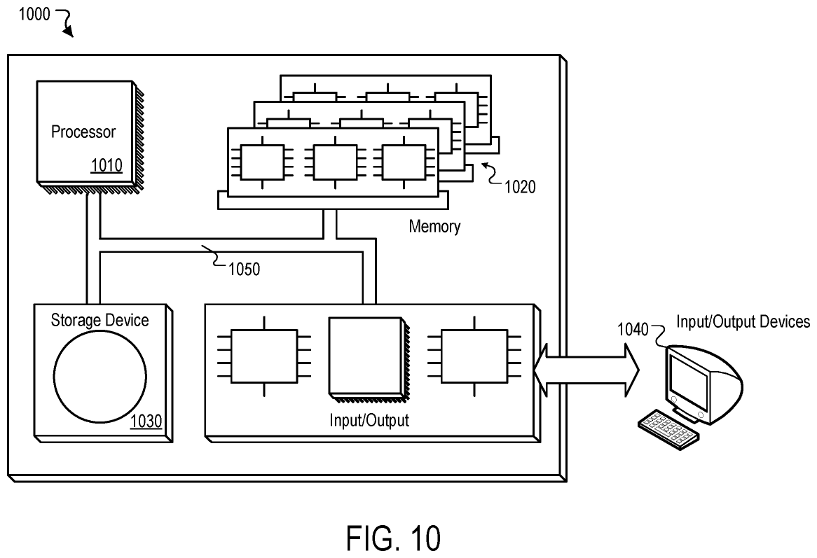

FIG. 10 is a schematic diagram of a computer system 1000. The system 1000 can be used to carry out the operations described in association with any of the computer-implemented methods described previously, according to some implementations. In some implementations, computing systems and devices and the functional operations described in this specification can be implemented in digital electronic circuitry, in tangibly-embodied computer software or firmware, in computer hardware, including the structures disclosed in this specification (e.g., system 1000) and their structural equivalents, or in combinations of one or more of them. The system 1000 is intended to include various forms of digital computers, such as laptops, desktops, workstations, personal digital assistants, servers, blade servers, mainframes, and other appropriate computers, including vehicles installed on base units or pod units of modular vehicles. The system 1000 can also include mobile devices, such as personal digital assistants, cellular telephones, smartphones, and other similar computing devices. Additionally, the system can include portable storage media, such as Universal Serial Bus (USB) flash drives. For example, the USB flash drives may store operating systems and other applications. The USB flash drives can include input/output components, such as a wireless transducer or USB connector that may be inserted into a USB port of another computing device.

The system 1000 includes a processor 1010, a memory 1020, a storage device 1030, and an input/output device 1040. Each of the components 1010, 1020, 1030, and 1040 are interconnected using a system bus 1050. The processor 1010 is capable of processing instructions for execution within the system 1000. The processor may be designed using any of a number of architectures. For example, the processor 1010 may be a CISC (Complex Instruction Set Computers) processor, a RISC (Reduced Instruction Set Computer) processor, or a MISC (Minimal Instruction Set Computer) processor.

In one implementation, the processor 1010 is a single-threaded processor. In another implementation, the processor 1010 is a multi-threaded processor. The processor 1010 is capable of processing instructions stored in the memory 1020 or on the storage device 1030 to display graphical information for a user interface on the input/output device 1040.

The memory 1020 stores information within the system 1000. In one implementation, the memory 1020 is a computer-readable medium. In one implementation, the memory 1020 is a volatile memory unit. In another implementation, the memory 1020 is a non-volatile memory unit.

The storage device 1030 is capable of providing mass storage for the system 1000. In one implementation, the storage device 1030 is a computer-readable medium. In various different implementations, the storage device 1030 may be a floppy disk device, a hard disk device, an optical disk device, or a tape device.

The input/output device 1040 provides input/output operations for the system 1000. In one implementation, the input/output device 1040 includes a keyboard and/or pointing device. In another implementation, the input/output device 1040 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, e.g., in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer. Additionally, such activities can be implemented via touchscreen flat-panel displays and other appropriate mechanisms.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

The computer system can include clients and servers. A client and server are generally remote from each other and typically interact through a network, such as the described one. The relationship of client and server arises by virtue of computer programs running on the respective computers and having a client-server relationship to each other.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

As used herein, the term “ready mix” refers to concrete that is batched for delivery from a central plant instead of being mixed on a job site. Typically, a batch of ready mix is tailor-made according to the specifics of a particular construction project and delivered in a plastic condition, usually in cylindrical trucks often referred to as “concrete mixers.”

As used herein, the term “real-time” refers to transmitting or processing data without intentional delay given the processing limitations of a system, the time required to accurately obtain data, and the rate of change of the data. Although there may be some actual delays, the delays are generally imperceptible to a user.

While this specification contains many specific implementation details, these should not be construed as limitations on the scope of what is being claimed, which is defined by the claims themselves, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claim may be directed to a subcombination or variation of a subcombination.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method of training a mixture prediction model, comprising:
obtaining input data including:
characterization data indicating characterizations of a plurality of ingredients,
environmental data indicating environmental conditions in which the plurality of ingredients are combined, and
recipe data defining a recipe for combining the plurality of ingredients;
mixing the plurality of ingredients according to the recipe to produce a concrete mixture;
obtaining sensor data representing one or more qualities of the concrete mixture;
evaluating the concrete mixture using the sensor data to obtain performance measures of the concrete mixture;
processing the input data with a mixture prediction model to obtain a corresponding output of the mixture prediction model, the corresponding output including predicted performance measures for the concrete mixture; and
adjusting parameters of the mixture prediction model based on comparing the output of the mixture prediction model to the performance measures of the concrete mixture.

2. The method of claim 1, wherein mixing the plurality of ingredients according to the recipe to produce the concrete mixture comprises:
determining, based on the recipe, a flow rate and a time for adding a first ingredient of the plurality of ingredients into a mixing vessel; and
controlling a flow of the first ingredient into the mixing vessel based on the determined flow rate and time.

3. The method of claim 1, wherein mixing the plurality of ingredients according to the recipe to produce the concrete mixture comprises:
determining, based on the recipe, a volume of a first ingredient to add to the concrete mixture; and
controlling a flow of the first ingredient to add the determined volume of the first ingredient to a mixing vessel.

4. The method of claim 1, comprising:
obtaining second input data including a second recipe for combining the plurality of ingredients;
mixing the plurality of ingredients according to the second recipe to produce a second mixture;
obtaining sensor data representing one or more qualities of the second mixture;
evaluating the second mixture using the sensor data to obtain performance measures of the concrete mixture;
processing the input data with the mixture prediction model to obtain a corresponding output of the mixture prediction model, the corresponding output including predicted performance measures for the second mixture; and
adjusting parameters of the mixture prediction model based on comparing the output of the mixture prediction model to the performance measures of the second mixture.

5. The method of claim 1, wherein obtaining sensor data representing one or more qualities of the concrete mixture comprises obtaining sensor data at time intervals during mixing of the concrete mixture.

6. The method of claim 1, wherein the environmental data indicates time-varying environmental conditions in which the plurality of ingredients are combined.

7. The method of claim 1, wherein the recipe indicates an amount of each ingredient of the plurality of ingredients to be mixed.

8. The method of claim 7, where the amount of each ingredient of the plurality of ingredients to be mixed comprises a proportion, a weight, volume, or a flow rate.

9. The method of claim 1, wherein the performance measures include at least one of slump or yield stress.

10. The method of claim 1, wherein the ingredients include particles, the characterizations including at least one of: particle size, particle shape, particle surface texture, porosity, particle chemical composition, and particle surface area.

11. The method of claim 1, wherein the plurality of ingredients include at least one of cement, water, fly ash, slag, plasticizer, fine aggregate, admixture, additives, and coarse aggregate.

12. The method of claim 1, wherein the environmental conditions include at least one of temperature, humidity, and time.

13. A method of generating a recipe for concrete, comprising:
obtaining input data including:
characterization data indicating characterizations of a plurality of ingredients,
environmental data indicating environmental conditions in which the plurality of ingredients are combined, and
recipe data defining a recipe for combining the plurality of ingredients to produce a concrete mixture;
predicting performance of the concrete mixture by processing the input data using a mixture prediction model configured to predict performance of the concrete mixture;
iteratively adjusting the recipe and predicting performance of the concrete mixture until the predicted performance satisfies performance criteria to obtain a final recipe; and
outputting the final recipe.

14. The method of claim 13, comprising:
mixing concrete using the final recipe; and
evaluating performance of the concrete using one or more performance tests.

15. The method of claim 13, wherein the ingredients include particles, the characterizations including at least one of: particle size, particle shape, particle surface texture, porosity, particle chemical composition, and particle surface area.

16. The method of claim 13, wherein the recipe indicates an amount of each ingredient of the plurality of ingredients to be mixed.

17. The method of claim 16, where the amount of each ingredient of the plurality of ingredients to be mixed comprises a proportion, a weight, volume, or a flow rate.

18. The method of claim 13, wherein predicting the performance of the concrete mixture comprises predicting a slump or yield stress of the concrete mixture.

19. The method of claim 13, wherein the plurality of ingredients include at least one of cement, water, fly ash, slag, plasticizer, fine aggregate, and coarse aggregate.

20. A system comprising one or more computers and one or more storage devices on which are stored instructions that are operable, when executed by the one or more computers, to cause the one or more computers to perform operations comprising:
obtaining input data including:
characterization data indicating characterizations of a plurality of ingredients,
environmental data indicating environmental conditions in which the plurality of ingredients are combined, and
recipe data defining a recipe for combining the plurality of ingredients;
mixing the plurality of ingredients according to the recipe to produce a concrete mixture;
obtaining sensor data representing one or more qualities of the concrete mixture;
evaluating the concrete mixture using the sensor data to obtain performance measures of the concrete mixture;
processing the input data with a mixture prediction model to obtain a corresponding output of the mixture prediction model, the corresponding output including predicted performance measures for the concrete mixture; and
adjusting parameters of the mixture prediction model based on comparing the output of the mixture prediction model to the performance measures of the concrete mixture.
